(19) United States
(12) Patent Application Publication
(10) Pub. No.: US 2010/0041905 A1
(43) Pub. Date: Feb. 18, 2010

(54) Process for the Preparation of Substituted 2-Acetylamino-Alkoxyphenyl

(75) Inventors: Debra Ainge, Loughborough (GB); Philip Cornwall, Loughborough (GB); Duncan Michael Gill, Loughborough (GB); Luis Manuel Vaz, Loughborough (GB)

Correspondence Address: FISH & RICHARDSON P.C., P.O BOX 1022, MINNEAPOLIS, MN, 55440-1022, US

(73) Assignee: ASTRAZENECA AB, Sodertalje (SE)

(21) Appl. No.: 12/374,230

(22) PCT Filed: Jul. 17, 2007
(86) PCT No.: PCT/SE2007/000693

§ 371 Date: Sep. 15, 2009

Related U.S. Application Data
(60) Provisional application No. 60/831,802, filed on Jul. 18, 2006.

(51) Int. Cl. C07D 303/00; C07C 233/00; C07C 43/00

(52) U.S. Cl. 549/513; 564/223; 568/584

(57) ABSTRACT

The present invention relates to a novel process for the preparation of compounds of formula (V) wherein X, Q, R1, R1a and R2 are as defined in the specification, the compounds being useful in the preparation of therapeutic agents.

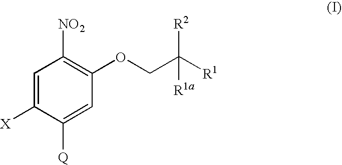

The present invention relates to novel processes for the preparation of intermediate compounds which can be used to prepare therapeutic agents. The present invention also relates to novel intermediate compounds which can be used to prepare therapeutic agents.

Chemokines play an important role in immune and inflammatory responses in various diseases and disorders, including asthma and allergic diseases, as well as autoimmune pathologies such as rheumatoid arthritis and atherosclerosis. Studies have demonstrated that the actions of chemokines are mediated by subfamilies of G protein-coupled receptors, among which are the receptors designated CCR1, CCR2, CCR2A, CCR2B, CCR3, CCR4, CCR5, CCR6, CCR7, CCR8, CCR9, CCR10 and CCR11 (for the C—C family); CXCR1, CXCR2, CXCR3, CXCR4 and CXCR5 (for the C—X—C family) and CX₃CR1 for the C—X₃—C family. These receptors represent good targets for drug development since agents which modulate these receptors would be useful in the treatment of disorders and diseases such as those mentioned above.

WO01/98273 discloses a series of compounds having a structure (IA) shown below, where R^{a} is a phenyl group (which may be substituted), where R^{b} represents a suitable substituent and n is typically 0, 1 or 2 and where R^{C} is hydrogen or a group such a C₁₋₆alkyl.

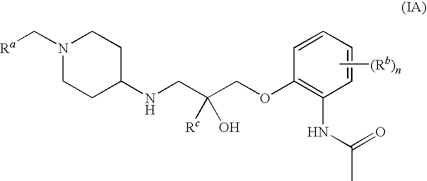

WO03/051839 discloses the CCR1 antagonist N-{2-[((2S)-3-{[1-(4-chlorobenzyl)piperidin-4-yl]amino}-2-hydroxy-2-methylpropyl)oxy]-4 hydroxyphenyl}acetamide. A related compound, N-{5-Chloro-2-[((2S)-3-{[1-(4-chlorobenzyl)piperidin-4-yl]amino}-2-hydroxy-2-methylpropyl)oxy]-4-hydroxyphenyl}acetamide has also been shown to antagonise CCR1 activity.

Methods of synthesising compounds of the type described above typically involve alkylation of a protected acetamidophenol derivative (2) with an epoxide derivative e.g. [2-methyloxiranyl]methyl-3-nitrobenzene sulfonate (3) (also known as methylglycidyl nosylate) to give an epoxy ether derivative (4) e.g. as shown in step (i) of scheme 1 below. Reaction of the epoxide product (4) with a piperidine amine (5) as shown in step (ii) of scheme 1 (and deprotection of any protected substituent groups) can give rise to the target pharmaceutical compound (1A).

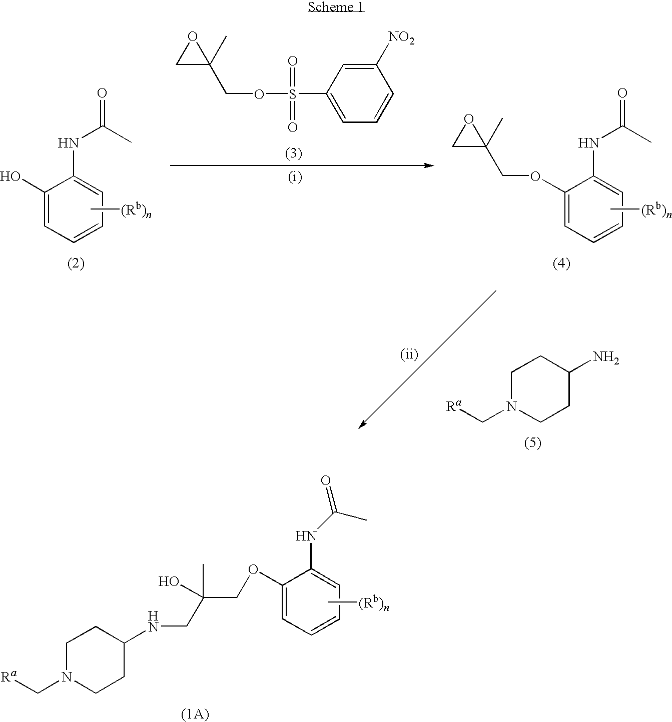

Whilst acceptable as a method to prepare target compounds in quantities of up to five kilograms, such routes are not considered suitable for further scale-up. One reason for this is the safety issues surrounding the transport and handling of the glycidyl nosylate (3), which has been found to have potentially dangerous thermal properties. Furthermore, known methods for the synthesis and purification of the glycidyl nosylate (3) can give rise to variable yields and significant levels of by-products.

In view of the above, it would be advantageous to find new methods of synthesising compounds of formula (IA).

The invention provides a process for preparing a compound of formula (I) or a salt thereof:

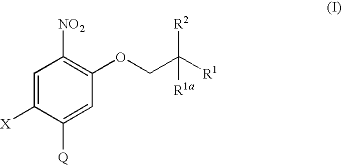

wherein Q is OH or OP where P is an alcohol-protecting group, or Q is fluorine or chlorine,
X is hydrogen or chlorine,
R¹ and R^{1a} together with the carbon atom to which they are attached form an epoxide ring group or R¹ and R^{1a} together form a precursor of an epoxide ring, and
R² is hydrogen or a C₁₋₃ alkyl group;
which process comprises reacting a compound of formula (II) or a salt thereof

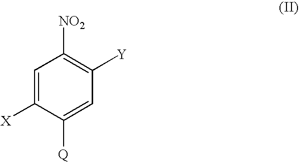

wherein Q and X are as defined in formula (I), and Y is chlorine or fluorine,
with a compound of formula (III) or a salt thereof

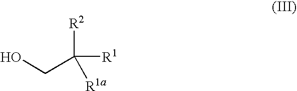

wherein R¹, R^{1a} and R² are as defined in relation to formula (I),
in the presence of a base;
and thereafter if desired, converting a group Q to a different group Q as defined above.

Unless otherwise indicated, the term ‘alkyl’ when used alone or in combination, refers to a straight chain or branched chain alkyl moiety. A C₁-C₆ alkyl group has from one to six carbon atoms including methyl, ethyl, n-propyl, isopropyl, tert-butyl, n-pentyl, n-hexyl and the like.

The process of the present invention is carried out in the presence of a base, typically an alkali metal base such as, but not limited to, potassium hydroxide, sodium hydroxide, sodium hydride, potassium hydride, potassium tert-butoxide, potassium tert-pentylate, potassium 3,7-dimethyl-3-octylate, butyl lithium, lithium di-isopropylamide, lithium hexamethyldisilazane or combinations thereof. In particular, the base may be a sterically hindered alkali metal alkoxide such as, but not limited to potassium tert-butoxide, potassium tert-pentylate and potassium 3,7-dimethyl-3-octylate.

The process of the present invention is suitably carried out in a solvent, for example a hydrocarbon, nitrile, polar aprotic or ether solvent. Suitable solvents include tetrahydrofuran, 2-methyl tetrahydrofuran, diethyl ether, di-isopropyl ether, acetonitrile, butyronitrile, N-methylpyrrolidinone, dimethylacetamide, dimethyl formamide, dimethyl sulfoxide, tert-butanol, toluene and xylenes, and combinations thereof. In one embodiment of the invention, the solvent is toluene.

Typically, the process is carried out at temperatures between −78° C. and 120° C., more preferably between −10° C. and 70° C. When Q is OH, the reaction is preferably carried out above 20° C. temperature, and when Q is OP or halogen, the reaction is preferably carried out at or below 20° C. temperature.

The nucleophilic aromatic substitution reaction (SnAr) process chemistry of the present invention is considered to give rise to a number of advantages. For example, the process of the present invention can be carried out using only a slight excess of a compound of formula (II). The process of the present invention can be volume efficient. Furthermore, the process of the invention allows for near stoichiometric quantities of compound of formula (II) and base. The SnAr approach of the present invention is simple to carry out, negating the need for metal catalysis or hazardous reagents. In particular, the process may be carried out without the use of potential genotoxic alkylating agents (e.g. chlorohydrins and sulfonate esters). The SnAr approach can also be carried out using cheap, readily available bases (such as potassium tert-butoxide). The process of the present invention can be operated in hydrocarbon, nitrile and ether solvents and may not necessarily require high boiling dipolar aprotics solvents such as dimethyl formamide, dimethyl sulfoxide and N-methyl pyrrolidinone. The SnAr approach of the present invention may also give rise to high yields and low levels of impurities. The SnAr approach also allows for relatively quick reactions.

Compounds of formula (I) in which Q is OH or OP can be prepared from compounds of formula (II) in which Q is OH or OP respectively. [In the case of OP, removal of the protecting group P is required at some later stage during the synthesis of the final product of formula (IA)]. However, when Q in formula (II) is OH and R¹ and R^{1a} together form a precursor to an epoxide group, in particular as described hereinafter, the process of the present invention can surprisingly be carried out without a protecting group to prepare a compound of formula (I) in which Q is OH. This can give rise to efficiency gains by negating the need for protection and deprotection steps.

The applicants have found also that groups Q may be changed for different such groups. In particular, compounds of formula (I) where Q is fluorine may be converted to groups of formula (I) where Q is hydroxy using hydroxide sources such as, but not limited to potassium hydroxide, sodium hydroxide, hydrogen peroxide, Triton B, tetrabutylammonium hydroxide, Aliquat 336, methyltributylammonium hydroxide or a combination thereof. Such reactions can be carried out at temperatures typically between 20-130° C. in solvents such as hydrocarbons (toluene), polar aprotic (dimethyl sulfoxide, dimethyl acetamide and N-methylpyrrolidinone) and alcohols (tert-butanol). Fluorine can be replaced with OH using a phase transfer catalyst, such as Triton B, tetrabutylammonium hydroxide, tetrabutylammonium bromide, Aliquat 336, methyltributylammonium chloride, methyltributylammonium hydroxide and an aqueous base, such as potassium hydroxide and sodium hydroxide and a solvent, such as hydrocarbons (toluene), polar aprotic (dimethyl sulfoxide, dimethyl acetamide and N-methylpyrrolidinone) and alcohols (tert-butanol). The reaction is advantageously carried out between 20-50° C.

In addition, OH can be introduced using reagents, that upon work-up liberate a free OH group. Such reagents include, but are not limited to, 2-butyn-1-ol (Synthetic Communications, 32 (9), 1401, 2002) and 2-(methylsulfonyl)ethanol (Tetrahedron Letters, 43, 3585, 2002).

In one embodiment of the process, R² is a C₁₋₃alkyl group. In particular R² is methyl.

In another embodiment, R² is hydrogen.

In one embodiment of the process of the invention, Y in formula (II) is fluorine.

In a further embodiment of the process of the invention, Q in formula (I) and formula (II) is OH or OP.

In a further embodiment of the process of the invention, Q in formula (I) and formula (II) is fluorine.

In a further embodiment of the process of the invention, X in formula (I) and formula (II) is hydrogen.

In a further embodiment of the process of the invention, X in formula (I) and formula (II) is chlorine.

In a further embodiment of the process of the invention, X in formula (I) and formula (II) is hydrogen or chlorine, Q is OH or OP, and Y is fluorine.

In a further embodiment of the process of the invention, X in formula (I) and formula (II) is hydrogen or chlorine, Q is fluorine and Y is fluorine.

In a further embodiment of the process of the invention, X in formula (I) and formula (II) is hydrogen or chlorine, Q is chlorine and Y is chlorine.

In a further embodiment of the process of the invention, X in formula (I) and formula (II) is hydrogen or chlorine, Q is chlorine and Y is fluorine.

Group Q in formula (I) and formula (II) may be OH or OP where P is an alcohol-protecting group.

The alcohol-protecting group P may in general be chosen from any of the groups described in the literature or known to the skilled chemist as appropriate for the protection of the group in question and may be introduced by conventional methods. The protecting group may be removed by any convenient method as described in the literature or known to the skilled chemist as appropriate for the removal of the protecting group in question, such methods being chosen so as to effect removal of the protecting group with minimum disturbance of groups elsewhere in the molecule. The protection and deprotection of hydroxy functional groups is well known in the art, and is described, for example, in ‘Protective Groups in Organic Chemistry’, edited by J. W. F. McOmie, Plenum Press (1973) and ‘Protective Groups in Organic Synthesis’, 3rd edition, T. W. Greene and P. G. M. Wutz, Wiley-Interscience (1999). Specific examples of protecting groups are given below for the sake of convenience, in which “lower”, as in, for example, lower alkyl, signifies that the group to which it is applied preferably has 1-4 carbon atoms. It will be understood that these examples are not exhaustive. Where specific examples of methods for the removal of protecting groups are given below these are similarly not exhaustive. The use of protecting groups and methods of deprotection not specifically mentioned are, of course, within the scope of the invention.

Examples of hydroxy protecting groups that may be used in the present invention include lower alkyl groups (for example tert-butyl), lower alkenyl groups (for example allyl); lower alkanoyl groups (for example acetyl); lower alkoxycarbonyl groups (for example tert-butoxycarbonyl); lower alkenyloxycarbonyl groups (for example allyloxycarbonyl); aryl-lower alkoxycarbonyl groups (for example benzyloxycarbonyl, 4-methoxybenzyloxycarbonyl, 2-nitrobenzyloxycarbonyl and 4-nitrobenzyloxycarbonyl); tri(lower alkyl)silyl (for example trimethylsilyl and tert-butyldimethylsilyl) and aryl-lower alkyl (for example benzyl) groups.

Typical protecting groups that may be used in the present invention include alkyl, allyl, acyl, benzyl, benzhydryl, trityl, or trialkylsilyl protecting groups. P may for example be methyl, ethyl, isopropyl, benzyl, p-methoxybenzyl or trityl; an alkoxyalkyl ether such as, but not limited to methoxymethyl; benzyl; or tetrahydropyranyl. The group OP may be an ester such as, but not limited to, acetate (i.e. P being acetyl) and benzoate. The group OP may be a silyl ether with P being, but not limited to, trimethylsilyl, triethylsilyl, tri-isopropylsilyl, tert-butyldimethylsilyl or tert-butyldiphenylsilyl.

In one aspect of the invention P is methyl, ethyl, isopropyl, benzyl, p-methoxybenzyl, trityl, methoxymethyl, tetrahydropyranyl acetyl, benzoate, trimethylsilyl, triethylsilyl, tri-isopropylsilyl, tert-butyldimethylsilyl or tert-butyldiphenylsilyl.

Compounds of formula (I), (II) and (III) may be in free base form or in salt form. The use of both free forms and salt forms are within the scope of the present invention. Salts may typically exist when Q in (I) and (II) is OH. Examples of salt forms include a base salt such as an alkali metal salt, for example lithium, sodium or potassium, or an alkaline earth metal salt, for example calcium or magnesium.

In particular, R¹ and R^{1a} together form a precursor of an epoxide group. Particular examples of such a precursor group is a group ═CH₂ or an oxo group ═O. Where R¹ and R^{1a} form an alkene group ═CH₂, this group can be converted directly into an epoxide group by epoxidation for example using an epoxidising agent such as m-chloroperoxybenzoic acid, peracetic acid, perbenzoic acid, trifluoroperacetic acid, magnesium monoperphthalate, tert-butyl hydroperoxide/vanadium, dimethyl dioxirane and manganese or cobalt salen complexes, or alternatively using epoxidase enzymes as outlined further below. Alternatively, it may be subject to a preliminary dihydroxylation step to form a group of sub-formula (i)

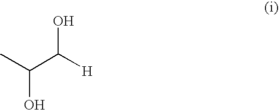

which may, in turn, be activated and converted to an epoxide group also as outlined further below.

Where R¹ and R^{1a} together form a ketone group of formula ═O, the compound may be converted to an epoxide group using conventional chemical methods, for instance using methylene transfer agents.

One example of such an agent is diazomethane, which may be reacted in organic solvents as described below, but in particular ethers, alcohols or chlorinated solvents.

Alternative methylene transfer agents include sulfur ylides which may be generated from reagents such as trimethylsulphonium iodide/chloride/bromide or fluoride, trimethylsulphoxonium iodide/chloride, dodecyldimethylsulphonium chloride, dimethyl sulphoxide, and a base, such as potassium tert-butoxide, potassium hydroxide, sodium hydroxide, sodium hydride or potassium carbonate with or without a phase transfer catalyst such as benzyltrimethylammonium chloride, cetyltrimethylammonium bromide and benzyltriethylammonium chloride.

Catalytic systems can also be employed, such as those that generate a ylide using a metallocarbene such as zinc or ruthenium carbenoids. In addition, use of a chiral sulfur ylide (such as those generated from camphorsulfonyl chloride) in both the stoichiometric or catalytic system can give rise to products with enhanced optical purity.

Reactions with methylene transfer agents are suitably conducted in an organic solvent. Suitable solvents include, but are not limited to nitrites (such as acetonitrile or butyronitrile), ethers (such as diethyl ether, methyl tert-butyl ether or tetrahydrofuran), alcohols (such as methanol, ethanol or isopropanol), polar aprotic solvents (such as dimethyl sulfoxide), chlorinated solvents (dichloromethane, chloroform, trichloroethane), hydrocarbons (such as toluene and hexane) or water.

Temperatures used will vary depending upon the particular reagents being used, but typically, temperatures of from −78° C. to 50° C., more preferably temperatures from zero to ambient will be used.

Thus, in a particular embodiment, the invention provides a process of preparing a compound of formula (IB) or a salt thereof:

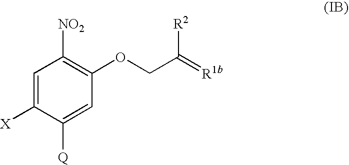

wherein Q is OH or OP where P is an alcohol-protecting group, or Q is fluorine or chlorine,
X is hydrogen or chlorine, R² is as defined in relation to formula (I) and R^{1b} is CH₂ or O, which process comprises reacting a compound of formula (II) as defined above, or a salt thereof, with a compound of formula (IIIB) or a salt thereof

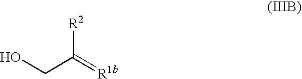

where R^{1b} is as defined in relation to formula (IB) and R² is as defined in relation to formula (I), in the presence of a base.

In an alternative embodiment, R¹ and R^{1a} together with the carbon atom to which they are attached form an epoxide group, so the compound of formula (I) is a compound of formula (IC)

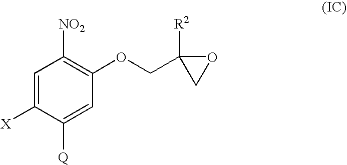

where X, Q and R² are as defined in relation to formula (I).

In this case, the compound of formula (III) is a compound of formula (IIIC)

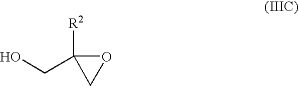

where R² is as defined in relation to formula (I).

In particular however, the compound of formula (IIIC) will be a stereospecific compound of formula (IIIC′)

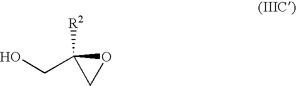

where R² is as defined in relation to formula (I), so that the resulting compound of formula (I) is also stereospecific and can be represented as (IC′)

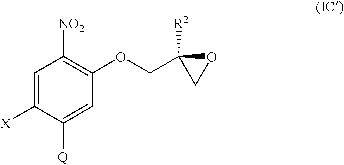

where X, Q and R² are as defined in relation to formula (I).

Where R¹ is a precursor group for an epoxide group, the nitro group may be reduced to an amine group and/or acylated before or after the precursor group R¹ is converted to an epoxide group to produce a compound of formula (4) as defined above.

Thus, the invention further provides a method for preparing a compound of formula (IV)

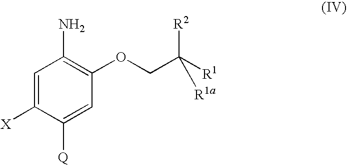

where R¹, R^{1a} and R² are as defined in relation to formula (I) which method comprises reduction of a compound of formula (I) as defined above.

The reduction is suitably carried out using known procedures for reducing the nitro group. Suitable reagents include, for example, ferrous salts such as ferrous sulfate and ferrous chloride and sodium dithionite. Moderate temperatures, for example from 0-60° C. and conveniently ambient temperature may be employed. The reaction is suitably carried out in a solvent such as water, aqueous ammonia or aliphatic alcohol and mixtures thereof.

Alternatively, the hydrogenation may be carried out using hydrogen and a catalyst such as a palladium, platinum or Raney Nickel catalyst such as 1-5% platinum on carbon. In this case, the reaction is suitably carried out at elevated pressures such as 1-60.0 bar pressure, for example at about 3 bar pressure in the presence of hydrogen. Temperatures in the range of from 20-70° C., for instance from 25-50° C. are suitably used. The reaction may be carried out in an organic solvent such as esters (such as but not limited to ethyl acetate and isopropyl acetate), acetic acid, water, alcohols (such as but not limited to methanol, ethanol, isopropanol), ethers (such as but not limited to diethyl ether, tetrahydrofuran and 2-methyl tetrahydrofuran) or a mixture thereof.

Compounds of formula (IV) may subsequently be acylated to form compounds of formula (V) or salts thereof.

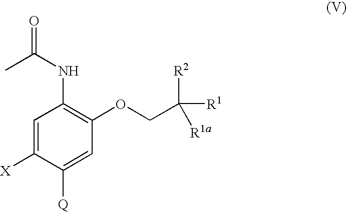

where R¹, R^{1a}, R², X and Q are as defined in relation to formula (I).

Suitable acylation conditions include reaction of the compound of formula (IV) with an acetyl halide such as acetyl chloride, or acetic anhydride. The reaction is suitably carried out in an organic solvent such esters (such as but not limited to ethyl acetate and isopropyl acetate), acetic acid, water, alcohols (such as but not limited to methanol, ethanol, isopropanol), ethers (such as but not limited to diethyl ether, tetrahydrofuran and 2-methyl tetrahydrofuran) or a mixture thereof. Moderate temperatures, for example from 0-60° C., and conveniently 20-25° C. are suitably employed.

Compounds of formula (IV) may be isolated prior to acylation, or they may be acylated in situ, for example by including acylating reagents in the hydrogenation reaction mixture.

Where Q is a hydroxy group, the acylation reaction may result in the conversion of the group OH to a group OP where P is an acetyl group. Where this occurs, deprotection as described above, for example by reaction with ammonia in an alkyl alcohol solvent such as methanol, will restore the OH group. Alternatively, deprotection can occur later in the synthesis.

As is clear from the above description, where R¹ is a precursor to an epoxide, it may be converted to an epoxide group at various stages.

Intermediates of formula (VII) form a further aspect of the invention.

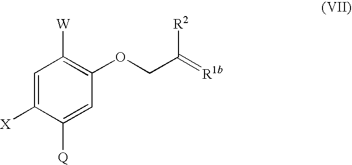

where R², X and Q are as defined in relation to formula (I) and W is NO₂, NH₂ or NHC(O)CH₃ and R^{1b} is CH₂ or O.

One embodiment of the invention relates to a compound of formula (IB) or a salt thereof

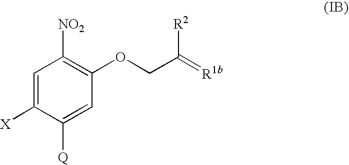

wherein Q is OH;
X is hydrogen or chlorine, R² is as defined in claim 1; and

### R^{1b} is CH₂ or O.

Another embodiment relates to the compounds 3-(2-Methyl-allyloxy)-4-nitro-phenol and 4-Amino-3-(2-methyl-allyloxy)-phenol.

A further embodiment relates to the compound of formula (IVB) or a salt thereof

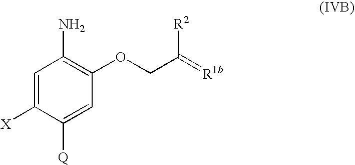

wherein Q is chlorine or fluorine;
X is hydrogen or chlorine;
R² is as defined in claim 1; and

### R^{1b} is CH₂ or O.

Yet further embodiment relates to a compound of formula (VB) or a salt thereof

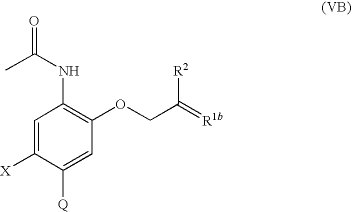

wherein Q is OH, OC(O)—CH₃, Q is chlorine or fluorine;
X is hydrogen or chlorine;
R² is as defined in claim 1; and

### R^{1b} is CH₂ or O.

One embodiment relates to compound acetic acid 4-acetylamino-3-(2-methyl-allyloxy)-phenyl ester and N-[4-Hydroxy-2-(2-methyl-allyloxy)-phenyl]-acetamide.

Another embodiment relates to compound (S)-Acetic acid 1-(2-acetylamino-5-hydroxy-phenoxymethyl)-2-bromo-1-methyl-ethyl ester.

Thus the invention further provides a method for preparing a compound of formula (VI) or a salt thereof

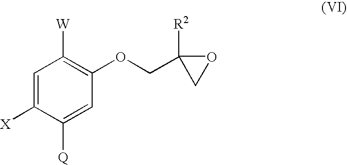

where R², X and Q are as defined in relation to formula (I) and W is NO₂, NH₂ or NHC(O)CH₃, which method comprises either
(A) reacting a compound of formula (VIIA) or a salt thereof

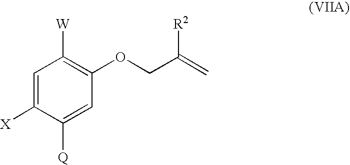

where R¹, X and Q are as defined in relation to formula (I) and W is as defined in relation to formula (VI), with an epoxidising agent, or
(B) reacting a compound of formula (VIIB) or a salt thereof

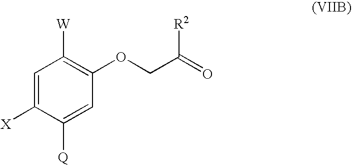

where R², X and Q are as defined in relation to formula (I) and W is as defined in relation to formula (VI), with a methylene transfer agent.

In the case of process (A) above, suitable epoxidising agents include m-chloroperoxybenzoic acid, peracetic acid, perbenzoic acid, trifluoroperacetic acid, magnesium monoperphthalate, tert-butyl hydroperoxide/vanadium, dimethyl dioxirane and manganese or cobalt salen complexes, or alternatively using epoxidase enzymes. The reaction is suitably carried out in an organic solvent such as chlorinated solvents (such as dichloromethane, carbon tetrachloride and 1,2-dichloroethane), non polar solvents (such as hexane, toluene and benzene), esters (such as ethyl acetate and isopropyl acetate), polar aprotic (such as dimethyl formamide) and aqueous mixtures thereof.

Moderate temperatures for example from 0 to 50° C., and conveniently ambient temperature, are suitably employed.

One embodiment of the invention relates to the compounds the compounds
3-(2-methyl-oxiranylmethoxy)-4-nitro-phenol,
Acetic acid 4-acetylamino-3-(2-methyl-oxiranylmethoxy)-phenyl ester,
2-(5-Fluoro-2-nitro-phenoxymethyl)-2-methyl-oxirane,
3-(2-Methyl-oxiranylmethoxy)-4-nitro-phenol,
Acetic acid 4-acetylamino-3-(2-methyl-oxiranylmethoxy)-phenyl ester,
3-(2-methyl-oxiranylmethoxy)-4-nitro-phenol, and
2-(5-Benzyloxy-2-nitro-phenoxymethyl)-2-methyl-oxirane.

In a further method (C), a compound of formula (VIII) or a salt thereof

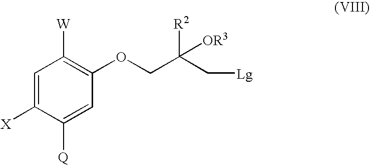

where R², X and Q are as defined in relation to formula (I), W is as defined in relation to formula (VI), R³ is hydrogen or a hydroxy protecting group, and Lg is a leaving group is reacted with a base.

In route (C) above, suitable examples of leaving groups Lg include sulfonate, tosylate, nosylate and mesylate as well as halide such as bromide. Suitable hydroxy protecting groups R³ include acetyl.

One embodiment relates to the compound (S)-Acetic acid 1-(2-acetylamino-5-hydroxy-phenoxymethyl)-2-bromo-1-methyl-ethyl ester.

The activated diols of formula (VIII) can be transformed to the epoxides upon treatment with a base using standard techniques. Suitable alkali metal bases include, but are not limited to, potassium carbonate, sodium hydroxide, potassium hydroxide, sodium hydride, sodium methoxide and sodium ethoxide.

Compounds of formula (VIII) may be obtained by activating compounds of formula (IX)

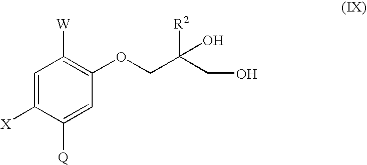

where R², X and Q are as defined in relation to formula (I) and W is as defined in relation to formula (VI).

Activation can be carried out using standard techniques (for example, tosyl, nosyl or mesyl chloride plus base respectively). Alternatively, the primary alcohol can be converted to the bromide using HBr or acetyl bromide in acetic acid to give the bromo acetoxy derivative [i.e. R³═CH₃C(O)—), and Lg=Br]. Upon treatment with base, the bromo acetoxy derivative forms the bromohydrin (i.e. R³═OH).

Compounds of formula (IX) are described and claimed in copending U.S. Application No. 60/799,574. In accordance with the present invention however, they may be prepared by dihydroxylation of a compound of formula (VIIA) as defined above.

Dihydroxylation conditions include reaction with a dihydroxylating agent such as a catalytic or stoichiometric osmium tetroxide or its equivalent (for example potassium osmate or osmium chloride). Due to the cost and toxicity of osmium compounds, it is preferable to use catalytic osmium reagent and a co-oxidant to regenerate the reagent. Such reagent include, but are not limited to, potassium hexacyanoferrate(III), hydrogen peroxide, sodium periodate, tert-butylhydrogen peroxide in the presence of tetra-n-butylammonium hydroxide or acetate, trimethylamine N-oxide in pyridine, N-methylmorpholine-N-oxide. Furthermore, addition of a chiral amine, such as dihydroquinidine or hydroquinone 1,4-phthalazinediyl, in the presence of a base such as an alkali metal carbonate, for instance potassium carbonate, (the so called Sharpless Asymmetric Dihydroxylation), can be used to produce diols with enhanced optical purity. Moderate temperatures for example from 0-40° C., and conveniently ambient temperature are employed.

The reaction may be carried out in a solvent such as water, alcohols (such as tert-butanol and isopropanol), chlorinated solvents (such as dichloromethane and carbon tetrachloride), non polar solvents (such as toluene and xylene), ethers (such as diethyl ether and methyl tert-butyl ether), nitrites (such as acetonitrile and butyronitrile), ketones (such as acetone and methyl isobutyl ketone), pyridine and mixtures thereof.

Compounds of formula (II) and (III) including (IIIC) above are known compounds or they can be prepared from known compounds by conventional methods.

A particular compound of formula (II) is a compound where Y is fluorine, X is chlorine and Q is hydroxyl. It has surprisingly been found that this compound may be prepared by nitration of 2-chloro-5-fluorophenol, for example as illustrated in Example 15 hereinafter. Although it might be expected that such a reaction would produce a mixture of isomers of the nitrated compound. It has now been found that the desired product 2-chloro-5-fluoro-4-nitrophenol is produced preferentially, and furthermore, that it may be crystallised out of solution, for example by addition of an antisolvent, and is readily isolable from other isomers.

One embodiment of the invention relates to a process for preparing a compound of formula (II), which is 2-chloro-5-fluoro-4-nitrophenol, which method comprises reacting 2-chloro-5-fluorophenol with a nitrating agent in an organic solvent, and crystallising the desired product from the solution. In another embodiment the crystallisation is effected by addition of an anti-solvent.

In a further embodiment the anti-solvent is n-heptane.

Compounds of formula (IB) and (VII), as well as compounds (IV) and (V) where R¹ and R^{1a} together form a ═CH₂ or ═O are novel (hereinafter referred to as compounds (IVB) and (VB) respectively), and so these and their salts, form a further aspect of the invention. In particular, R¹ and R^{1a} together form a ═CH₂ group.

Compounds of formula (VI) (VIII) and (IX) are described and claimed in copending U.S. Patent Application No. 60/799,574.

Certain compounds used in the methods of the invention are capable of existing in stereoisomeric forms, and it will be understood that the invention encompasses all optical isomers of the compounds of formula (I) and mixtures thereof including racemates.

Thus in accordance with the present invention, the key intermediates of formula (4) above, can be prepared efficiently without using toxic intermediates.

For clarity, the various ways of achieving this using the method of the invention is summarised in Schemes 2 and 3

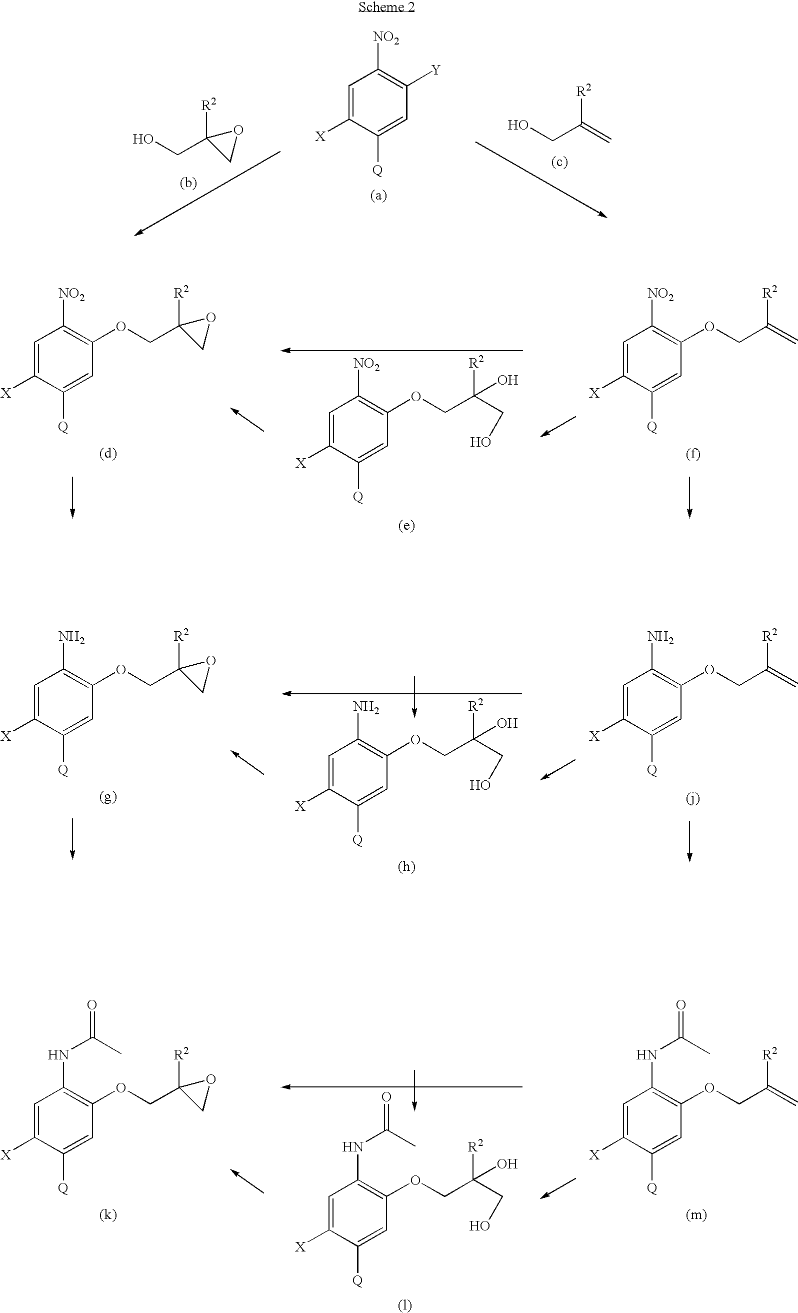

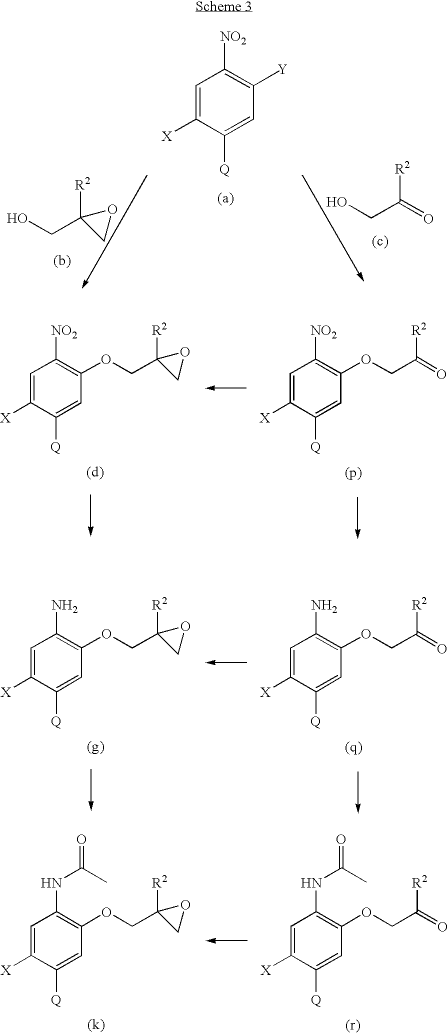

In schemes 2 and 3, R², X and Q are as defined in relation to formula (I). In addition, compounds (e), (h) and (l) in Scheme 2 may be converted to compounds (d), (g) and (k) respectively via the appropriate activated intermediates of formula (VIII) as outlined above.

Epoxide compounds obtained using the method of the invention, and in particular compound (k) can be converted to target CCR1 antagonists of formula (IA) above (where R^{a} is a phenyl group, which may be substituted, for example as referred to in WO01/98273) by reaction with a piperidine amine as shown in scheme 1, using analogues methods to those described in WO01/98273.

The invention will now be further explained with reference to the following illustrative examples.

Unless otherwise specified, all starting materials and reagents were purchased from standard suppliers (Sigma Aldrich, Apollo, Johnson Matthey and Fisher Scientific), and were used without further purification unless otherwise stated. Reactions were carried out using standard glassware under a nitrogen atmosphere, unless otherwise stated.

NMR spectra were acquired on Varian Inova 300 MHz or 400 MHz or Bruker 300 MHz and 200 MHz spectrometers (as detailed) as solutions in suitably deuterated solvents. Nominal masses were determined either by GCMS or LCMS (as detailed). LCMS were ran on an Agilent binary 1100 HPLC with 80 Hz DAD and Multimode ES+APCl positive ion, Agilent LCMS DSL (negative ion) or a Waters 2790 HPLC equipped with 996 Photo Diode Array detector and Micromass ZMD (single quadropole mass spectrometer with Z-spray interface). GCMS data was acquired using an Agilent 6890 GC coupled to a 5973 MSD, equipped with either EI or CI source. For CI experiments, reagent grade methane from BOC gases was used as reagent gas. Chiral HPLC was ran on an Agilent HP-1100 VWD Detector.

### EXAMPLE 1

### 4-Fluoro-2-(2-methyl-allyloxy)-1-nitro-benzene

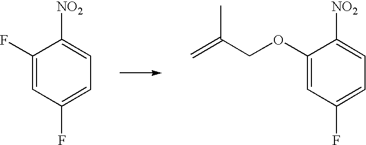

Potassium tert-butoxide (4.27 mmol; 493.73 mg) and toluene (8.00 ml) were charged to a flask. A solution of methallyl alcohol (1.10 eq; 6.71 mmol; 579.40 μl; 493.48 mg) in toluene (2.00 ml) was charged and the contents of the vessel were left to stir for 30 min. The solution was cooled to −5° C. A solution of 2,4-difluoronitrobenzene (6.10 mmol; 688.71 μl; 1.00 g) in toluene (4.00 ml) was added and left to stir at −5° C. for 1 h. A second charge of potassium tert-butoxide (1.83 mmol; 211.60 mg) was added and the mixture continued to stir at −5-0° C. for 3 h. A third charge of potassium tert-butoxide (609.71 μmol; 70.53 mg) was added and the reaction stirred for an additional 30 min. Water (5.00 ml) was added and the two layers were separated. The organic layer was washed with water (5.00 ml) and concentrated in vacuo to give an oil that was titurated with pentane (7.00 ml) to give the title compound in 75% yield.

¹H NMR (300 MHz, DMSO): δ 8.04 (dd, J=9.1, 6.1 Hz, 1H), 7.31 (dd, J=11.1, 2.6 Hz, 1H), 6.98 (ddd, J=9.0, 7.9, 2.5 Hz, 1H), 5.12 (s, 1H), 5.01 (s, 1H), 4.69 (s, 2H), 1.78 (s, 3H). GC-MS (CI) m/z 240 (M+C₂H₅⁺), 212 (MH⁺), 166 (MH⁺−NO₂), 140 (M−OCH₂C(CH₃)CH₂).

### EXAMPLE 2

### 4-Chloro-2-(2-Methyl-Allyloxy)-1-Nitro-Benzene

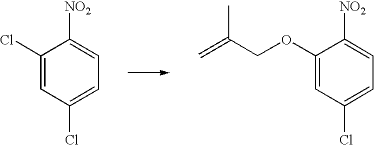

Methallyl alcohol (23.62 mmol; 2.00 ml; 1.70 g) was charged to a mixture of 2,4-dichloro-1-nitrobenzene (1.00 eq; 23.62 mmol; 4.54 g) and potassium hydroxide (23.62 mmol; 1.33 g) in isopropyl alcohol (8.52 ml; 6.70 g) and water (8.52 ml). The mixture was heated at reflux. Ater 16 h at reflux, methallyl alcohol (23.62 mmol; 2.00 ml; 1.70 g) was added and heating continued. After an additional 16 h, potassium hydroxide (23.62 mmol; 1.33 g) and methallyl alcohol (23.62 mmol; 2.00 ml; 1.70 g) were added and heating continued overnight. The reaction was cooled to ambient and water (20 ml) was added. The resulting solution was extracted with EtOAc (100 ml) and the organic phase was concentrated in vacuo to give the crude product. The crude product was slurried in 25 ml toluene at reflux, cooled, filtered and dried overnight to give the title compound as an orange solid in 32% yield.

¹H NMR (299.947 MHz, DMSO) δ 7.96 (m, 1H), 7.49 (d, J=2.1 Hz, 1H), 7.21 (m, 1H), 5.08 (s, 1H), 5.00 (s, 1H), 4.75 (s, 2H), 1.77 (d, J=5.6 Hz, 3H). GCMS m/z 229 (MH⁺), 182 (MH⁺−NO₂).

### EXAMPLE 3

### 3-(2-Methyl-allyloxy)-4-nitro-phenol

### Method 1

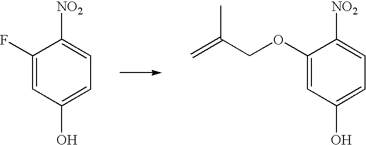

Potassium tert-butoxide (954.80 mmol; 107.14 g) and 2-methyltetrahydrofuran (250.00 ml) were charged to a flask. Methallyl alcohol (636.53 mmol; 53.89 ml; 45.90 g) and 2-methyltetrahydrofuran (100.00 ml) were added at rt. The reaction exothermed to 43° C. 3-Fluoro-4-nitrophenol (318.27 mmol; 50.00 g) was dissolved in 2-methyltetrahydrofuran (150.00 ml) and added dropwise over 1 h to the reaction mixture. The reaction was heated to reflux. After 2 days water (500 ml) and 37% w/w HCl (50 ml) were added to give an aqueous phase with pH 5-6. The two phases were separated. The organic layer was concentrated to dryness to give the title compound in 85% yield.

### Method 2

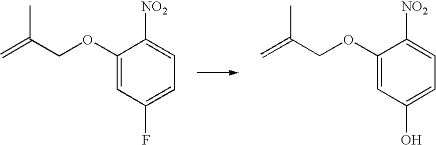

To a 50 ml three necked flask was charged 4-fluoro-2-(2-methyl-allyloxy)-1-nitro-benzene (4.74 mmol; 1.00 g), dimethyl sulfoxide (10.00 ml) and potassium hydroxide (50% w/w, 14.21 mmol; 1.65 ml; 1.99 g). The reaction was heated to 40° C. for 2 h. The reaction was cooled to room temperature. Water (8.00 ml) was charged and the pH adjusted to 6 using glacial acetic acid. The product was extracted into ethyl acetate (8.00 ml), washed with water (8.00 ml), dried with magnesium sulphate and concentrated in vacuo. The resulting solid was slurried in pentane (10.00 ml) and the resulting yellow solid was collected by filtration to give the title compound in 61% yield.

¹H NMR (399.819 MHz, DMSO) δ 10.83 (d, J=21.3 Hz, 1H), 7.90 (q, J=4.4 Hz, 1H), 6.58 (m, 1H), 6.48 (d, J=2.3 Hz, 1H), 5.14 (s, 1H), 4.99 (s, 1H), 4.58 (s, 2H), 1.78 (s, 3H).

LCMS (ESI) m/z 232 (MH⁺+Na⁺), 210 (MH⁺), 192 (M−OH), 164 (MH⁺−NO₂)

### EXAMPLE 4

### 3-(5-Hydroxy-2-nitro-phenoxy)-2-methyl-propane-1,2-diol

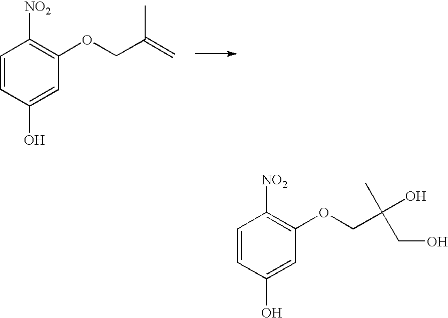

Water (15.00 ml), potassium carbonate (14.34 mmol; 1.98 g), potassium hexacyanoferrate(III) (14.34 mmol; 4.77 g), potassium osmate (VI) dihydrate (239.00 μmol; 88.06 mg) and hydroquinine 1,4-phthalazinediyl diether (121.95 μmol; 100.00 mg) were added to a 50 ml three necked flask. 3-(2-Methyl-allyloxy)-4-nitro-phenol (4.78 mmol; 1.00 g) in tert-butyl alcohol (15.00 ml) was added and the reaction was stirred at room temperature over the weekend. After this time, a second charge of potassium osmate (VI) dihydrate (239.00 μmol; 88.06 mg) was added and stirring continued overnight. Sodium metabisulfite (29.34 mmol; 5.75 g) in water (11.50 ml) was added dropwise. Ethyl acetate (15.00 ml; 13.51 g) was added and the two layers separated. The organic layer was then washed sequentially with water (9 ml), sulfuric acid 2 M (6 ml), sodium bicarbonate (9 ml) and brine (9 ml). The organic phase was concentrated to give the title compound in 65% yield.

¹H NMR (399.819 MHz, DMSO) δ 10.83 (s, 1H), 7.88 (d, J=9.2 Hz, 1H), 6.56 (d, J=2.6 Hz, 1H), 6.45 (dd, J=9.0, 2.3 Hz, 1H), 3.93 (d, J=9.0 Hz, 1H), 3.78 (d, J=9.0 Hz, 1H), 3.36 (m, 2H), 1.14 (s, 3H). LCMS (ESI) m/z 266 (MH⁺+Na⁺), 244 (MH⁺), 226 (MH⁺−H₂O)

### EXAMPLE 5

### 3-(2-methyl-oxiranylmethoxy)-4-nitro-phenol

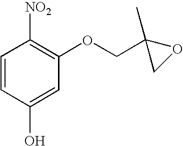

To a 50 ml 3-neck flask was added the isopropyl acetate solution of acetic acid 1-(2-nitro-5-hydroxy-phenoxymethyl)-2-bromo-1-methyl-ethyl ester (9.5 ml, 6.6 mmol). The mixture was cooled to −5° C. and 25% w/w sodium methoxide in methanol (3.7 ml, 16.24 mmol) was added dropwise. The reaction was allowed to progress at ambient temperature. After 30 min the reaction was quenched with water (10 ml). The biphasic mixture was separated and acetic acid (0.61 ml, 10.6 mmol) was added to the aqueous phase. The aqueous solution was extracted with isopropyl acetate (20 ml). The organic solution was concentrated in vacuo to yield the title product in 69% yield. ¹H-NMR (299.947 MHz, DMSO) δ 10.90 (s, 1H), 7.91 (d, J=9.0 Hz, 1H), 6.58 (s, 1H), 6.49 (d, J=9.0 Hz, 1H), 4.14 (dd, J=10.8, 85.1 Hz, 2H), 2.80 (dd, J=5.4, 43.8 Hz, 2H), 1.40 (s, 3H). m/z LCMS (ESI+ve) 226 (MH⁺).

### EXAMPLE 6

### 4-Amino-3-(2-methyl-allyloxy)-phenol

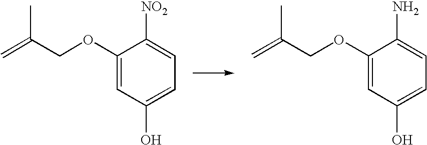

3-(2-Methyl-allyloxy)-4-nitro-phenol (0.5 g, 2.39 mmol) in water (8 ml) was reduced using sodium dithionite (10.76 mmol; 1.87 g). After 1 h at room temperature 2 M HCl was added to pH 1 (to destroy excess reagents, 30 ml), followed by 40% NaOH to pH 5. During the addition of NaOH a solid precipitated. This was isolated by filtration and dried in a vacuum oven overnight to give the title compound in 95% yield.

¹H NMR (299.944 MHz, DMSO) δ 8.45 (s, 1H), 6.48 (dd, J=8.2, 2.3 Hz, 1H), 6.30 (t, J=2.2 Hz, 1H), 6.15 (dt, J=8.4, 2.4 Hz, 1H), 5.02 (d, J=37.6 Hz, 2H), 4.38 (s, 2H), 1.80 (s, 3H). LCMS m/z 180 (MH⁺).

### EXAMPLE 7

### Acetic acid 4-acetylamino-3-(2-methyl-allyloxy)-phenyl ester

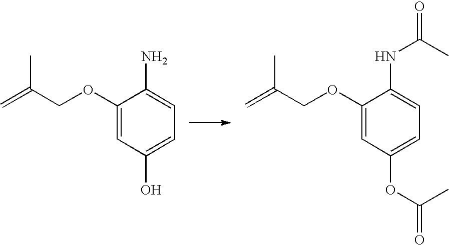

4-Amino-3-(2-methyl-allyloxy)-phenol (5.58 mmol; 1.00 g) was dissolved in 2-methyltetrahydrofuran (10.00 ml) and triethylamine (16.74 mmol; 2.33 ml). Acetyl chloride (16.74 mmol; 1.19 ml) was added dropwise at room temperature. After 2 h, the reaction was quenched with water and the organic phase was separated. The organic phase was concentrated to give the title compound in 85% yield.

¹H NMR (399.819 MHz, DMSO) δ 9.09 (s, 1H), 7.74 (d, J=8.6 Hz, 1H), 6.81 (d, J=1.9 Hz, 1H), 6.65 (dd, J=8.6, 2.3 Hz, 1H), 5.07 (s, 1H), 4.96 (s, 1H), 4.50 (s, 2H), 2.24 (s, 3H), 2.07 (s, 3H), 1.78 (s, 3H). LCMS m/z 286 (MH⁺+Na⁺), 264 (MH⁺).

### EXAMPLE 8

### N-[4-Hydroxy-2-(2-methyl-allyloxy)-phenyl]-acetamide

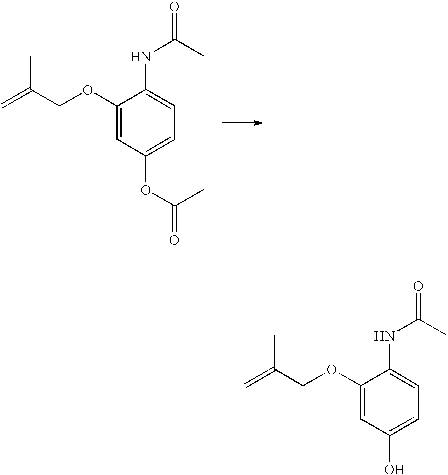

To acetic acid 4-acetylamino-3-(2-methyl-allyloxy)-phenyl ester (10.03 g, 1 eq) was added methanol (200 ml). The solution was heated to 45° C. Ammonia in methanol (6 ml, 7 M, 1.1 eq) was added. The reaction was allowed to progress at 45° C. for 2 h before being cooled to ambient and stirred overnight. The reaction was acidified and product partitioned between water and ethyl acetate. Solvent was removed under vacuum to give the title compound in 90% yield.

¹H NMR (399.819 MHz, DMSO) δ 9.25 (s, 1H), 8.81 (s, 1H), 7.34 (d, J=8.7 Hz, 1H), 6.38 (d, J=2.6 Hz, 1H), 6.28 (dd, J=8.5, 2.6 Hz, 1H), 5.06 (s, 1H), 4.94 (s, 1H), 4.41 (s, 2H), 2.00 (d, J=11.3 Hz, 3H), 1.76 (s, 3H). LCMS m/z 222 (MH⁺), 180 (M−COCH₃).

### EXAMPLE 9

### Acetic acid 4-acetylamino-3-(2,3-dihydroxy-2-methyl-propoxy)-phenyl ester

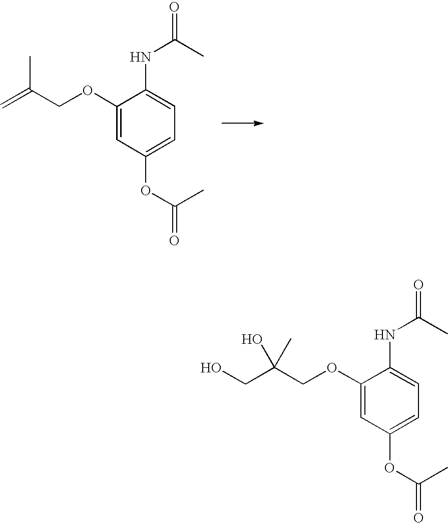

Acetic acid 4-acetylamino-3-(2-methyl-allyloxy)-phenyl ester (2.66 mmol; 700.00 mg) was added to a mixture of potassium carbonate (7.98 mmol; 1.10 g), hydroquinidine 1,4-phthalazinediyl diether (26.59 μmol; 20.71 mg), potassium hexacyanoferrate (III) (7.98 mmol; 2.63 g) and potassium osmate (VI) dihydrate (13.29 μmol; 4.90 mg) in water (21.00 ml) and tert-butyl alcohol (21.00 ml) at room temperature. After 3 h at room temperature the reaction was quenched by addition of sodium sulfite (15.95 mmol; 2.01 g) in water (20 ml). The reaction was extracted with isopropyl acetate (20 ml). The organic phase was concentrated to dryness to give the title compound in 72% yield.

¹H NMR (399.817 MHz, CDCl₃) δ 8.35 (d, J=8.7 Hz, 1H), 7.88 (s, 1H), 6.72 (m, 2H), 4.12 (d, J=10.8 Hz, 1H), 3.97 (d, J=11.0 Hz, 1H), 2.78 (d, J=4.6 Hz, 1H), 2.92 (d, J=4.6 Hz, 1H), 2.27 (s, 3H), 2.23 (s, 3H), 1.48 (s, 3H). LCMS m/z 320 (MH⁺+Na⁺), 298 (MH⁺).

### EXAMPLE 10

### Acetic acid 4-acetylamino-3-(2-methyl-oxiranylmethoxy)-phenyl ester

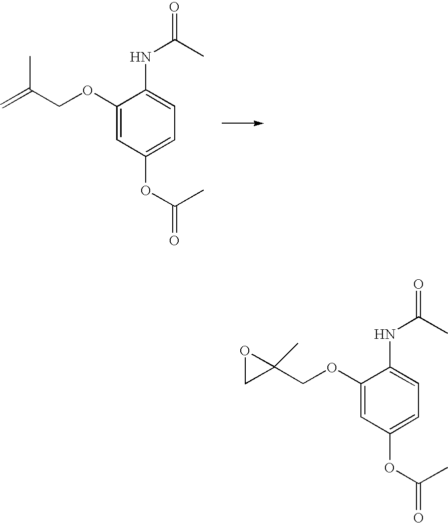

m-Chloroperoxybenzoic acid (4.18 mmol; 1.03 g) in dichloromethane (10.00 ml) was added at room temperature to a solution of acetic acid 4-acetylamino-3-(2-methyl-allyloxy)-phenyl ester (3.80 mmol; 1.00 g) in dichloromethane (10.00 ml). The reaction was stirred at room temperature overnight. 2 M NaOH was added (10 ml) and the two phases were separated. The organic phase was concentrated to give the title compound in 34% yield.

1H NMR (299.947 MHz, DMSO) δ 9.07 (s, 1H), 7.71 (m, 1H), 6.86 (d, J=2.5 Hz, 1H), 6.68 (dd, J=8.6, 2.5 Hz, 1H), 4.16 (d, J=11.1 Hz, 1H), 3.90 (d, J=11.1 Hz, 1H), 2.84 (d, J=5.0 Hz, 1H), 2.70 (m, 1H), 2.33 (s, 3H), 2.05 (d, J=16.3 Hz, 3H), 1.32 (d, J=49.9 Hz, 3H). LCMS m/z 302 (MNa⁺).

### EXAMPLE 11

### N-[2-(2,3-Dihydroxy-2-methyl-propoxy)-4-hydroxy-phenyl]-acetamide

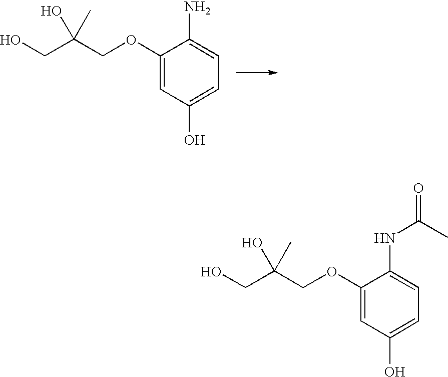

3-(2-Amino-5-hydroxy-phenoxy)-2-methyl-propane-1,2-diol (1.3 g) was dissolved in isopropanol (26 ml) at ambient temperature. Acetic anhydride (0.86 ml) was added and the mixture heated to 60° C. for 1 h, then stirred overnight at ambient. The solvent was removed in vacuo to leave the title compound in 90%.

¹H NMR (399.826 MHz, DMSO) δ 9.22 (s, 1H), 8.85 (s, 1H), 7.55 (d, J=8.5 Hz, 1H), 6.39 (d, J=2.6 Hz, 1H), 6.28 (dd, J=8.6, 2.4 Hz, 1H), 4.74 (m, 2H), 3.78 (m, 1H), 3.68 (d, J=9.0 Hz, 1H), 3.45 (dd, J=10.6, 5.5 Hz, 1H), 3.25 (m, 1H), 2.02 (s, 3H). LCMS m/z 256 (MH⁺), 238 (MH⁺−H₂O), 220 (MH⁺−2H₂O)

### EXAMPLE 12

### 2-(5-Fluoro-2-nitro-phenoxymethyl)-2-methyl-oxirane

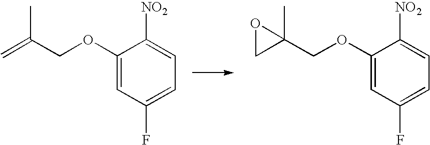

m-Chloroperoxybenzoic acid (5.21 mmol; 1.17 g) in dichloromethane (10.00 ml) was added to 4-fluoro-2-(2-methyl-allyloxy)-1-nitro-benzene (4.74 mmol; 1.00 g) in dichloromethane (10.00 ml) at room temperature. After 4 h m-chloroperoxybenzoic acid (5.21 mmol; 1.17 g) was charged. The reaction was stirred overnight at room temperature. Sodium hydroxide (2 M, 20 ml) was charged and the two layers were separated. The organic layer was concentrated to give the title compound in 25% yield.

¹H NMR (300 MHz, DMSO): δ 8.04 (dd, J=9.1, 6.1 Hz, 1H), 7.31 (dd, J=11.1, 2.6 Hz, 1H), 7.00 (ddd, J=9.1, 7.8, 2.5 Hz, 1H), 4.40 (d, J=10.8 Hz, 1H), 4.12 (d, J=10.8 Hz, 1H), 2.83 (d, J=4.8 Hz, 1H), 2.73 (d, J=4.8 Hz, 1H), 1.39 (s, 3H). GCMS (CI) m/z 256 (M+C₂H₅⁺), 228 (MH⁺), 208 (M−F), 158 (MH⁺−CH₂C(CH₃)(OCH₂))

### EXAMPLE 13

### 3-(2-Methyl-oxiranylmethoxy)-4-nitro-phenol

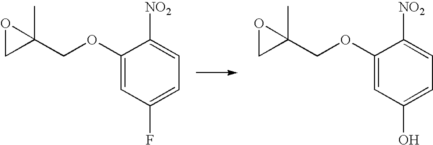

To a 50 ml three necked flask were charged 2-(5-fluoro-2-nitro-phenoxymethyl)-2-methyl oxirane (1.00 eq 8.80 mmol; 2.00 g), dimethyl sulfoxide (20.00 ml) and 50% w/w potassium hydroxide (22.01 mmol; 2.56 ml; 3.09 g). The resulting mixture was heated up to 40° C. After 1.5 h water (16.00 ml) was charged and the pH was adjusted to 6 using glacial acetic acid. Ethyl acetate (16.00 ml) was charged and the two layers separated. The aqueous layer was washed twice more with ethyl acetate (16.00 ml) and the combined organic layers were concentrated to dryness to give the title compound in 71% yield.

¹H NMR (300 MHz, DMSO): δ 10.95 (s, 1H), 7.90 (d, J=9.0 Hz, 1H), 6.57 (d, J=2.3 Hz, 1H), 6.49 (dd, J=9.0, 2.3 Hz, 1H), 4.28 (d, J=10.8 Hz, 1H), 4.00 (d, J=10.8 Hz, 1H), 2.87 (d, J=4.8 Hz, 1H), 2.72 (d, J=5.1 Hz, 1H), 1.40 (s, 3H). LCMS (ESI) m/z 248 (MH⁺+Na⁺), 226 (MH⁺), 180 (MH⁺−NO₂), 138 (M−OCH₂C(CH₃)CH₂(O))

### EXAMPLE 14

### (S)-Acetic acid 1-(2-acetylamino-5-hydroxy-phenoxymethyl)-2-bromo-1-methyl-ethyl ester

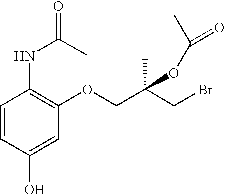

Hydrobromic acid in acetic acid (42.5 ml, 3 equiv.) was added to (S)—N-[2-(2,3-dihydroxy-2-methyl-propoxy)-4-hydroxy-phenyl]-acetamide (20 g) in acetic acid (40 ml) at 40° C. The reaction was heated at 40° C. for approximately 2 h. Isopropyl acetate (200 ml) was added followed by water. The aqueous phase was removed and the organic layer was washed sequentially with ammonium hydroxide solution and sodium sulfite solution. The product can be isolated by concentration to dryness. Alternatively, the solution can be used directly in the next stage.

### EXAMPLE 15

### Acetic acid 4-acetylamino-3-(2-methyl-oxiranylmethoxy)-phenyl ester

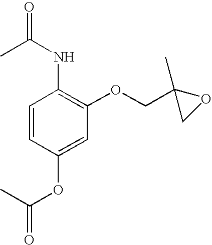

### Method 1

Sodium methoxide (41.2 ml, 2.3 equiv.) was added to acetic acid 1-(2-acetylamino-5-hydroxy-phenoxymethyl)-2-bromo-1-methyl-ethyl ester (approx 78 mmol, 160 ml), at −10° C. After 30 min at this temperature, acetic anhydride (10 ml, 1.35 mol equiv.) was added at −5° C. This reaction was stirred for 30 min then quenched by addition of water. The two phases were separated and the organic phase was washed with sodium bicarbonate solution. The organic phase was concentrated by distillation, then diluted with heptane (40 ml). The solution was cooled to induce crystallisation, and the title compound was isolated by filtration.

### Method 2

To an hydrogenation reactor were charged 3-(2-methyl-oxiranylmethoxy)-4-nitro-phenol (5 g, 22.2 mmol), isopropyl acetate (50 ml), triethylamine (9.3 ml, 66.6 mmol), acetic anhydride (7.4 ml, 77.5 mmol) and 1% platinum on charcoal (22.6 μmol Pt, 1 g, 55.9% water). The mixture was stirred at 25° C. under 4 barg of hydrogen. After complete hydrogenation, the reaction mixture was filtered on buchner to remove the catalyst. The organic solution was washed with sodium carbonate and brine. The washed organic solution was concentrated in vacuo to yield the title compound in 97% yield. ¹H-NMR (299.947 MHz, DMSO) δ 9.1 (s, 1H), 7.70 (d, J=8.7 Hz, 1H), 6.90 (d, J=2.4 Hz, 1H), 6.70 (dd, J=2.4, 8.4 Hz, 1H), 4.05 (m, 2H), 2.80 (m, 2H), 2.3 (s, 3H), 2.1 (s, 3H), 1.40 (s, 3H). m/z LCMS (ESI+ve) 280.2 (MH⁺), 262.2 (MH⁺−H₂O), 220.2 (MH⁺−H₂O—CH₃CO).

### EXAMPLE 16

### 2-Chloro-5-fluoro-4-nitrophenol

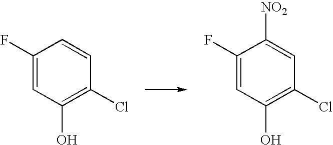

Ferric nitrate nonahydrate (14.06 g; 98% w/w; 34 mmol) was added to a solution of 2-chloro-5-fluorophenol (5.0 g; 34 mmol) in ethanol (125 ml). The resulting mixture (containing suspended solid) was stirred and heated to 50-55° C. and maintained in this temperature range for 4 to 5 h, by which time the suspended solid was almost completely dissolved. Analysis by HPLC revealed complete reaction of the starting material. The mixture was cooled to 25-30° C. and water (50 ml) was added. The mixture was then extracted with chloroform (3×25 ml) and the combined chloroform extracts washed with water (2×25 ml). The chloroform layer was evaporated under reduced pressure at 35° C. Toluene (15 ml) was added to the residue and heated to 50-55° C. and maintained within that temperature range for 10 min to give a clear solution. n-Heptane was slowly added to the solution, maintaining the temperature at 50-55° C. Crystallisation was observed during the n-heptane addition. The resulting slurry was stirred at 50-55° C. for 30 min then slowly cooled to 30-35° C. The mixture was filtered at this temperature and the collected solid washed with n-heptane (15 ml). The product was dried in vacuo at 30-35° C. to give the title compound as a fluffy solid in 45% yield.

¹H-NMR (200.13 MHz, CDCl₃) δ 8.21 (d, J=7.4 Hz, 1H), 6.95 (d, J=11.4 Hz, 1H), 6.27 (br.s, 1H). LCMS (ES⁻) m/z 190 (M−H)⁻

### EXAMPLE 17

### 4-Benzyloxy-2-fluoro-1-nitro-benzene

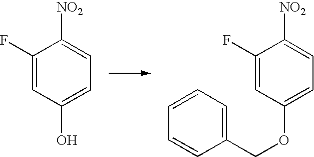

3-Fluoro-4-nitrophenol (127.31 mmol; 20.00 g) was dissolved in dimethylformamide (200.15 ml). Potassium carbonate (254.61 mmol; 35.19 g) was added. Benzylbromide (127.31 mmol; 15.18 ml; 21.77 g) was added at room temperature and the reaction was stirred overnight. Diethyl ether (250 ml) and water (250 ml) were added. The two phases were separated and the aqueous phase was extracted with diethyl ether (2×250 ml). The organic layers were combined, washed with 20% w/w brine (4×125 ml), dried over MgSO₄ and concentrated to give the title compound in 92% yield.

¹H NMR (299.946 MHz, CDCl₃) δ 8.11 (m, 1H), 7.45 (m, 5H), 6.77 (m, 2H), 5.18 (d, J=19.2 Hz, 2H). LCMS m/z 248 (MH⁺).

### EXAMPLE 18

### 2-(5-Benzyloxy-2-nitro-phenoxymethyl)-2-methyl-oxirane

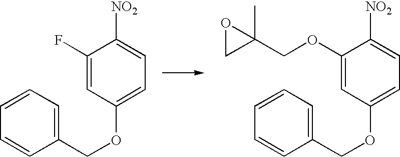

Potassium tert-butoxide (60.67 mmol; 7.17 g) was slurried in toluene (37.5 ml) at room temperature. A solution of glycidol (1.05 eq; 63.71 mmol; 5.79 g) in toluene (37.5 ml) was added between 10-20° C. Tetrahydrofuran (15.00 ml) was added to aid dissolution. This solution was transferred to a 100 ml dropping funnel, filtered through a cotton wool plug and added to 4-benzyloxy-2-fluoro-1-nitro-benzene (60.67 mmol; 15.00 g) in toluene (75 ml) between 3-8° C. In an additional flask, Glycidol (0.20 eq; 12.13 mmol; 1.10 g) in tetrahydrofuran (10.00 ml) was added at room temperature to potassium tert-butoxide (12.13 mmol; 1.43 g) in tetrahydrofuran (10 ml). The resulting solution was added to the reaction mixture. The reaction was stirred for 2 h. Water (150 ml) and tert-butyl methyl ether (200 ml) were added. The two phases were separated. The aqueous phase was extracted with tert-butyl methyl ether (2×150 ml). The organic layers were combined, washed with brine, dried over MgSO₄ and evaporated to give the title compound in 69% yield.

¹H NMR (399.817 MHz, CDCl₃) δ 7.98 (dd, J=9.1, 5.3 Hz, 1H), 7.40 (m, 5H), 6.63 (d, J=2.3 Hz, 1H), 6.59 (dd, J=9.1, 2.4 Hz, 1H), 5.20 (s, 2H), 4.15 (d, J=10.5 Hz, 1H), 4.01 (d, J=10.5 Hz, 1H), 2.96 (d, J=4.6 Hz, 1H), 2.75 (d, J=4.6 Hz, 1H), 1.51 (s, 3H).

LCMS m/z 316 (MH⁺).

### EXAMPLE 19

### 3-(2-Amino-5-hydroxy-phenoxy)-2-methyl-propane-1,2-diol

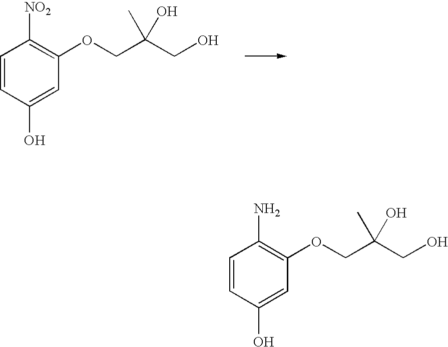

3-(5-Hydroxy-2-nitro-phenoxy)-2-methyl-propane-1,2-diol (2.5 g, 10.28 mmol) was charged to a mixture of ethyl acetate (37.5 ml) and 5% Pd/C catalyst (0.5 g, 20% w/w). The mixture was hydrogentated at 5 barg and room temperature overnight. The reaction mixture was filtered. The resulting solution was concentrated in vacuo to give the title compound in 61% yield.

¹H NMR (299.947 MHz, DMSO) δ 8.38 (s, 1H), 6.42 (d, J=8.2 Hz, 1H), 6.25 (d, J=2.3 Hz, 1H), 6.11 (dd, J=8.2, 2.1 Hz, 1H), 4.62 (s, 2H), 3.30 (m, 5H). LCMS m/z 236 (MH⁺+Na⁺), 214 (MH⁺).

1. A process for preparing a compound of formula (I) or a salt thereof 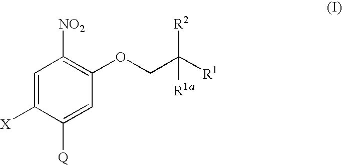
wherein Q is OH or OP where P is an alcohol-protecting group, or Q is fluorine or chlorine,
X is hydrogen or chlorine,
R¹ and R^{1a} together with the carbon atom to which they are attached form an epoxide ring group or R¹ and R^{1a} together form a precursor of an epoxide ring, and
R² is hydrogen or a C₁₋₃ alkyl group,
which process comprises reacting a compound of formula (II) or a salt thereof 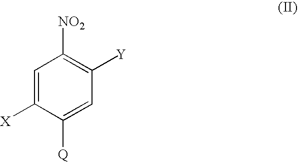
wherein Q and X are as defined in formula (I), and Y is chlorine or fluorine,
with a compound of formula (III) or a salt thereof 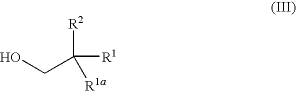
wherein R¹, R^{1a} and R² are as defined in relation to formula (I),
in the presence of a base;
and thereafter if desired, converting a group Q to a different group Q as defined above.

2. A process according to claim 1 wherein R¹ and R^{1a} together form a precursor of an epoxide group of formula ═CH₂ or ═O.

3. A process according to claim 2 wherein R¹ and R^{1a} together form a ═CH₂ group.

4. A process according to claim 2 wherein R¹ and R^{1a} together form a ═O group.

5. A process according to claim 1 wherein R² is methyl.

6. A process according to claim 1 wherein R² is hydrogen.

7. A process according to claim 1, wherein Y is fluorine.

8. A process according to claim 1, wherein Q is OH.

9. A process according to claim 1, wherein Q is fluorine.

10. A process according to claim 1, wherein X is hydrogen.

11. A process according to claim 1, wherein X is chlorine.

12. A process according to claim 1, wherein X is hydrogen, Q is OH or OP, and Y is fluorine.

13. A process according to claim 1, wherein X is hydrogen, Q is fluorine and Y is fluorine.

14. A process according to claim 9, wherein the group Q is subsequently converted to an OH group.

15. A process according to claim 1, wherein P is methyl, ethyl, isopropyl, benzyl, p-methoxybenzyl, trityl, methoxymethyl, tetrahydropyranyl acetyl, benzoate, trimethylsilyl, triethylsilyl, tri-isopropylsilyl, tert-butyldimethylsilyl or tert-butyldiphenylsilyl.

16. A process according to claim 1 wherein the compound of formula (I) is subsequently reduced to form a compound of formula (IV) or a salt thereof 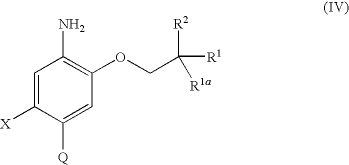
where X, Q, R¹, R^{1a} and R² are as defined in claim 1.

17. A process according to claim 16 wherein the compounds of formula (IV) is subsequently acylated to form a compound of formula (V) or a salt thereof 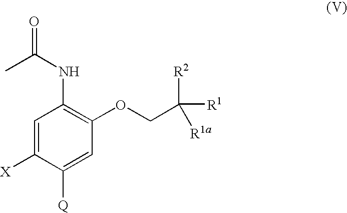
wherein X, Q, R¹, R^{1a} and R² are as defined in claim 1.

18. A process according to claim 1 wherein the compound of formula (I) is subsequently reduced and acylated in-situ to form a compound of formula (V) or a salt thereof 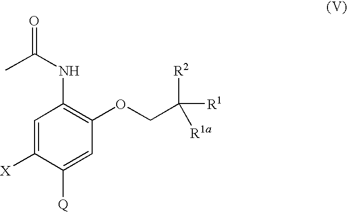
where X, Q, R¹, R^{1a} and R² are as defined in claim 1.

19. A process for preparing a compound of formula (VI) or a salt thereof 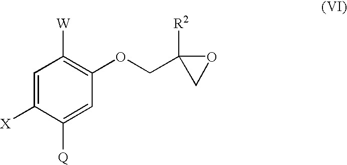
where R², X and Q are as defined in claim 1 and W is NO₂, NH₂ or NHC(O)CH₃, which method comprises either
(A) reacting a compound of formula (VIIA) 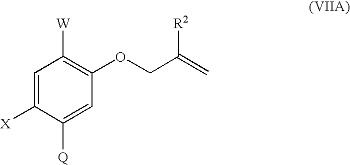
where R¹, X and Q are as defined in claim 1 and W is as defined above with an epoxidising agent,
(B) reacting a compound of formula (VIIB) or a salt thereof 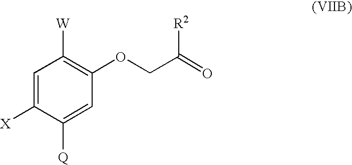
where R², X and Q are as defined in relation to formula (I) and W is as defined in relation to formula (VIIA), with a methylene transfer agent.

20. The compounds
3-(2-methyl-oxiranylmethoxy)-4-nitro-phenol,
Acetic acid 4-acetylamino-3-(2-methyl-oxiranylmethoxy)-phenyl ester,
2-(5-Fluoro-2-nitro-phenoxymethyl)-2-methyl-oxirane,
3-(2-Methyl-oxiranylmethoxy)-4-nitro-phenol,
Acetic acid 4-acetylamino-3-(2-methyl-oxiranylmethoxy)-phenyl ester,
3-(2-methyl-oxiranylmethoxy)-4-nitro-phenol, and
2-(5-Benzyloxy-2-nitro-phenoxymethyl)-2-methyl-oxirane.

21. A compound of formula (IB) or a salt thereof 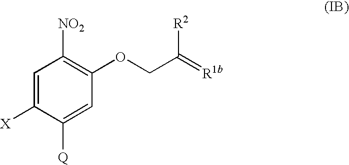
wherein Q is OH;
X is hydrogen or chlorine, R² is as defined in claim 1; and
R^{1b} is CH₂ or O.

22. The compound 3-(2-Methyl-allyloxy)-4-nitro-phenol.

23. A compound of formula (IVB) or a salt thereof 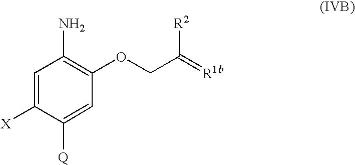
wherein Q is chlorine or fluorine;
X is hydrogen or chlorine;
R² is as defined in claim 1; and
R^{1b} is CH₂ or O.

24. The compound 4-Amino-3-(2-methyl-allyloxy)-phenol.

25. A compound of formula (VB) or a salt thereof 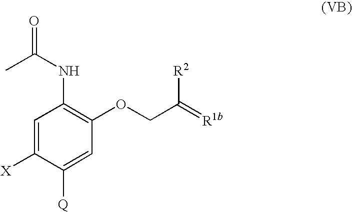
wherein Q is OH, OC(O)—CH₃, Q is chlorine or fluorine;
X is hydrogen or chlorine;
R² is as defined in claim 1; and
R^{1b} is CH₂ or O.

26. The compounds acetic acid 4-acetylamino-3-(2-methyl-allyloxy)-phenyl ester and N-[4-Hydroxy-2-(2-methyl-allyloxy)-phenyl]-acetamide.

27. A compound (S)-Acetic acid 1-(2-acetylamino-5-hydroxy-phenoxymethyl)-2-bromo-1-methyl-ethyl ester.

28. A compound according to claim 21, wherein X is hydrogen.

29. A compound according to claim 10, where R^{1b} is ═CH₂.

30. A compound according to claim 10 where R^{1b} is ═O.

31. A compound according to claim 10 where R² is methyl.

32. A compound according to claim 10 where R² is hydrogen.

33. A process for preparing a compound of formula (IX) 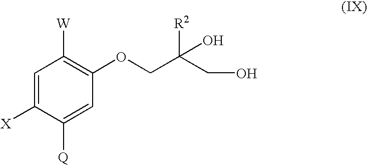
where R², X and Q are as defined in claim 1 and W is NO₂, NH₂ or NHC(O)CH₃, which comprises dihydroxylation of a compound of formula (VII) 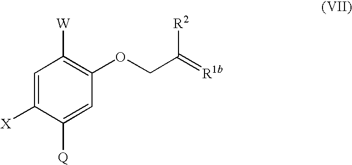
wherein Q is OH or OP where P is an alcohol protecting group, or Q is chlorine or fluorine;
X is hydrogen or chlorine;
R^{1b} is CH₂ or O;
R² is CH₂; and
W is NO₂, NH₂ or NHC(O)CH₃.

34. A compound according to claim 21 wherein P is methyl, ethyl, isopropyl, benzyl, p-methoxybenzyl, trityl, methoxymethyl, tetrahydropyranyl acetyl, benzoate, trimethylsilyl, triethylsilyl, tri-isopropylsilyl, tert-butyldimethylsilyl or tert-butyldiphenylsilyl.

35. A process for preparing a compound of formula (II) according to claim 1, which method comprises reacting 2-chloro-5-fluorophenol with a nitrating agent in an organic solvent, and crystallising the desired product from the solution.

36. The process according to claim 34 wherein the crystallisation is effected by addition of an anti-solvent.

37. The process according to claim 34 wherein the anti-solvent is n-heptane.
